(19) United States
(12) Patent Application Publication
(10) Pub. No.: US 2026/0355512 A1
(43) Pub. Date: Nov. 12, 2026

(54) SYSTEMS AND METHODS FOR NEUROSTIMULATION FOR APPLICATIONS IN WOMEN'S HEALTH CONDITIONS

(71) Applicant: SAMPHIRE NEUROSCIENCE LTD, London (GB)

(72) Inventors: Emile Radyte; Alexander Cook, London (GB); Ervinas Bernatavicus, London (GB)

(21) Appl. No.: 19/475,290

(22) PCT Filed: Apr. 12, 2024
(86) PCT No.: PCT/GB2024/050975

§ 371 Date: Oct. 14, 2025

(30) Foreign Application Priority Data
Apr. 14, 2023 (US) 63496384
Apr. 12, 2024 (WO) PCT/GB2024/050975

(51) Int. Cl. A61N 1/36; A61N 1/04

(52) U.S. Cl. CPC A61N 1/36021; A61N 1/0456; A61N 1/0472

(57) ABSTRACT

A system and method for neurostimulation for menstrual cycle symptom relief that can include a neurostimulation device that may have two zones of electrodes and a management application. The system and method may also include receiving stimulation input data; determining a stimulation profile based on the stimulation input data; and activating a neurostimulation device according to the stimulation profile, which may be adapted for stimulation session schedule management, using feedback to dynamically adjust stimulation, and calibrate use of a neurostimulation device.

### CROSS-REFERENCE TO RELATED APPLICATIONS

This Application is a 371 National Stage Application of PCT Application No. PCT/GB2024/050975, filed on 12 Apr. 2024, which claims the benefit of U.S. Provisional Application No. 63/496,384, filed on 14 Apr. 2023, both of which a incorporated in their entirety by this reference.

### TECHNICAL FIELD

This invention relates generally to the field of neurostimulation, and more specifically to a new and useful system and method for a neurostimulation for applications in women's health conditions, especially with regards to menstrual cycle symptom relief, especially with regards to applications in women's health conditions.

### BACKGROUND OF THE INVENTION

Neurostimulation is a field of technology that uses electrical stimulation for altering or modulating a subject's nervous system. The field of neurostimulation has been explored using invasive and non-invasive approaches.

However, neurostimulation devices face many challenges hindering broader adoption. Current solutions often require manual calibration by professionals at clinical sites, limiting accessibility. Additionally, the need for precise calibration restricts device use, especially during physical activity. Tailoring neurostimulation to individual neuroanatomy is also complex due to costly imaging equipment and uncertainties about optimal brain targets.

On another topic, there are many challenges in the women's health space. Symptoms and conditions experienced by women are often overlooked or solely treated with medication. Women experiencing menstrual cycle symptoms, such as severe period pains and anxiety, may not find adequate relief with conventional medications and may encounter undesirable side effects. This highlights the need for alternative, more effective treatment options tailored to women's health needs.

Thus, there is a need in the neurostimulation field to create a new and useful system and method for neurostimulation for women's health conditions, especially with regards to menstrual cycle symptom relief. This invention provides such a new and useful system and method.

### BRIEF DESCRIPTION OF DRAWINGS

FIG. 1 is a schematic representation of a system variation.

FIG. 2A is a schematic representation of dual arc variation of a neurostimulation device from a perspective view.

FIG. 2B is a schematic representation of dual arc variation of a neurostimulation device from a bottom view.

FIG. 3 is a schematic representation of a dual arc variation of a neurostimulation device with one discontinuous arc.

FIG. 4 is a schematic representation of exemplary screenshots of a management application during a variation of position calibration.

FIG. 5 is a schematic representation of a variation of a network of electrodes arrangement.

FIG. 6 is a schematic representation of another variation of a network of electrodes arrangement.

FIG. 7 is a detailed schematic representation of one electrode design variation.

FIG. 8 is a flowchart representation of a method variation.

FIG. 9 is a flowchart representation of a method variation using input data.

FIG. 10 is a flowchart representation of a method variation scheduling stimulation sessions based on menstrual cycle data.

FIG. 11 is a flowchart representation of a method variation for receiving stimulation feedback data.

FIG. 12 is a flowchart representation of a method variation scheduling stimulation sessions based on menstrual cycle data while incorporating calibration and feedback data.

FIG. 13 is a flowchart representation of a method variation of calibrating position of a neurostimulation device based on detected sensations.

FIG. 14 is a flowchart representation of a method variation of calibrating position of a neurostimulation device based on electrode contact.

FIG. 15 is a flowchart representation of a method variation of calibrating position for electrode stimulation steering.

FIG. 16 is a schematic representation of exemplary regions targeted by different electrode groups in a dual-zone neurostimulation device mapped to a 10-20 EEG system.

FIG. 17 is an exemplary system architecture that may be used in implementing the system and/or method.

### DETAILED DESCRIPTION OF THE EMBODIMENTS

The following description of the embodiments of the invention is not intended to limit the invention to these embodiments but rather to enable a person skilled in the art to make and use this invention.

### 1. Overview

Described herein are systems and methods for neurostimulation for women's health treatment. In particular, the systems and methods may use dynamic neurostimulation for relief during menstrual cycles.

The systems and methods may be used in controlling operation of a neurostimulation device that may be used by subjects directly, potentially outside of clinical settings and potentially without direct oversight by treatment professionals. Accordingly, the systems and methods described herein include technical enhancements enforcing proper application of neurostimulation (e.g., scheduling, preventing over-use, etc.), automatically personalizing operation of a neurostimulation device for enhanced performance, and/or adapting operation of a neurostimulation device based on patterns of subject use (e.g., compensating for device positioning).

The systems and methods, in some variations, may apply a novel neurostimulation device with multiple targeted zones of stimulation. In particular, the neurostimulation device may have two zones of targeted stimulation. In other words, the neurostimulation device may be a dual-zone neurostimulation device. This may be particularly useful in treatment of menstrual cycle symptoms by being able to apply neuromodulation to regions associated with mood (e.g., the dorsolateral prefrontal cortex or DLPFC) and pain (e.g., the motor cortex or MC).

In some variations, the system and methods may include variations for dynamically positioned neurostimulation, which may use a stimulation approach that can enable digitally controlled targeting or steering of stimulation. The systems and methods are preferably used so that a worn device can target location of stimulation in precise locations, enabling better control and/or enhanced response.

In particular, the systems and methods may utilize an array of electrodes that can be dynamically controlled such that an effective stimulation region can be positioned by the system without needing the whole device to be precisely manipulated. In some variations, the systems and methods may be used so that an array of anode and cathode electrodes are selectably activated (and/or deactivated) so that the resulting stimulation (e.g., current/potential differences) is shifted to different positions relative to the worn headset frame.

The systems and methods may include an array of different electrodes of at least one polarity. In such a situation, a set of electrodes may be selectably activated to simulate a first electrode polarity promoting stimulation with a second electrode of the opposite polarity. In this way, different combinations of active electrodes can create different stimulation positions. In other variations, the stimulation positioning may be more finely adjusted by using combined proportional stimulation of a set of electrodes such that a virtual/simulated electrode position is established.

In some variations, the dynamic positioning of targeted neurostimulation may be used to direct stimulation at multiple targeted regions. For example, the stimulation allows two targeted zones to be targeted despite variations in exact device positioning.

The systems and methods may additionally or alternatively enable advancements in the field of neurostimulation as it relates to calibration and/or the design of stimulation hardware, as well as in understanding the neurocognitive effects of more expanded or precise targeting of neurostimulation, especially in the fields of women's health. The application of this invention in women's health is also novel and is enabled by the fact that effective at-home neurostimulation, enabled through the systems and methods herein, allows for the management of symptoms for common women's health conditions, previously too inaccessible with neurostimulation alternatives. In-clinic transcranial electrical stimulation (TES) and transcranial magnetic stimulation (TMS) treatment is not feasible for most normal people who would not have time or resources to use such treatment, especially for health conditions like menstrual cycle symptoms that require regular reoccurring treatment. Furthermore, such professionally operated solutions do not offer the automated management and operation capabilities to be used directly by a subject.

The systems and methods may include variations enabling enhanced calibration of the placement of a worn position, which may enable neurostimulation to be used in situations where subjects can use the device without oversight from a trained expert.

The systems and methods may include variations that utilize alternative electrode designs that are specifically designed for improved usability for a subject. In particular, the alternative electrode design may have enhancements that enable neurostimulation devices to be used by subjects with different hair styles that previously presented challenges when trying to implement neurostimulation.

The systems and methods are preferably used to create a more usable and effective therapeutic solution. In general, the systems and methods are preferably employed within a neurostimulation device used for modulating neurological activity of a subject. The system and method may be particularly helpful in treating or modulating symptoms relating to pain, mood, and/or cognition. In some preferred applications, the systems and methods may have use in the management of symptoms in women's health conditions. In particular, the systems and methods may be used to manage or provide relief for premenstrual syndrome (PMS), primary and/or secondary dysmenorrhea, premenstrual dysphoric disorder (PMDD), polycystic ovary syndrome (PCOS), endometriosis, and/or other menstrual cycle related health conditions.

In some instances, the systems and methods may improve mood and reduce anxiety symptoms associated with the menstrual cycle. In some instances, the systems and methods may ameliorate or reduce pain symptoms associated with the menstrual cycle. In yet other instances, the systems and methods may reduce fatigue and/or result in improvement of cognitive functionality associated with the menstrual cycle. Herein, the systems and methods are primarily described as they may be used to provide relief for health concerns related to women's and individuals assigned female at birth's menstrual cycle. However, the systems and methods may additionally have applications in other health conditions, especially in the women's health space such as postpartum depression and menopause. The systems and methods may also have applications in the more general health space such as in the treatment for general psychological or psychiatric conditions and/or the promotion of neuroplasticity.

Herein, subject is used to refer to the wearer of the device and the receiver of the neurostimulation, which can be a person.

The system and method may provide a number of potential benefits. The system and method are not limited to always providing such benefits and are presented only as exemplary representations for how the system and method may be put to use. The list of benefits is not intended to be exhaustive and other benefits may additionally or alternatively exist.

As one potential benefit, the systems and methods may provide dynamic planning of stimulation. This may include dynamically planning a schedule of stimulation sessions. This may additionally or alternatively include adjusting stimulation configuration to personalize and enhance results for a subject. This adjustment may be based on feedback from previous sessions and/or based on positioning of the neurostimulation devices.

As another potential benefit, the systems and methods may enable a unique a neurostimulation device that is able to target two (or more) distinct brain regions. This may provide targeted neuromodulation across two zones. In the field of women's health, this may help address different symptoms, which at times may both be present and interdependent.

As another potential benefit, the systems and methods may be used in enabling dynamically adjusted stimulation positioning. In some variations, the systems and methods may enable a solution that can avoid needing to have physical fine-tuned placement of electrodes on a subject. In some variations, the systems and methods can digitally adjust the created stimulation field after the headset frame is roughly positioned in an appropriate region.

As another potential benefit, the systems and methods may enable a more usable device. The systems and methods may include the combination of multiple features such that a subject could use a neurostimulation device outside of a clinical setting and potentially without the assistance of another person assisting in the placement of electrodes. For example, the dynamical control over stimulation positioning can relieve users from needing to precisely position electrodes on their head. After approximate positioning of a headset is achieved, the electrodes can be dynamically controlled to establish more precise stimulation.

As a related potential benefit, the systems and methods may enable feedback mechanisms (both manual and/or automatic) such that a user could more easily calibrate positioning of a device.

As another potential benefit, the systems and methods of some variations may use an alternative electrode design. For example, the systems and methods may make use of a comb electrode design. Such an electrode design may be more usable—affording the possibility for a subject to put on a headset on their own. The electrode design may also be more effective by wearers with thicker hair such as females with longer hair.

### 2. System

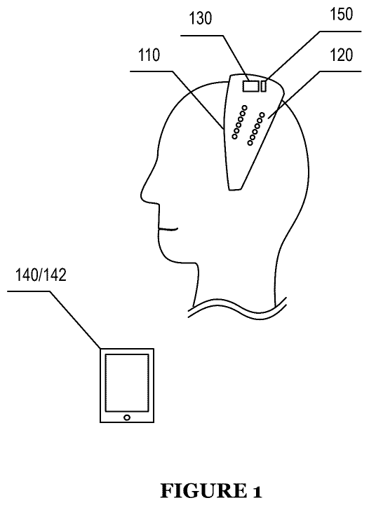

As shown in FIG. 1, a system for a neuromodulation may can include a neurostimulation device that includes headset frame 110, a plurality of electrodes 120 integrated into the headset frame, and control circuitry 130 electrically coupled (or connectable) to the electrodes. In some variations, the system may additionally include a control user interface 140 which may be or include a management application 142, which may be used to facilitate calibration and/or operation of the neurostimulation device. In some variations, the system may additionally or alternatively include a position sensing system 150.

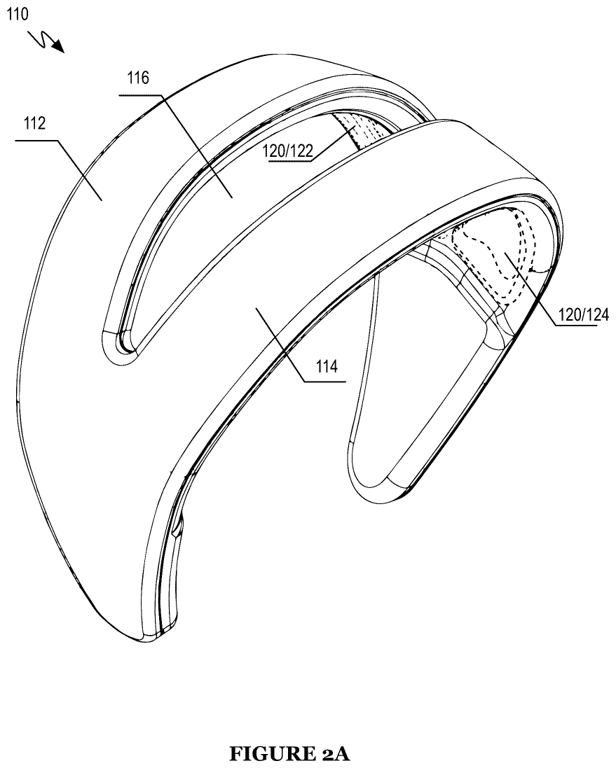

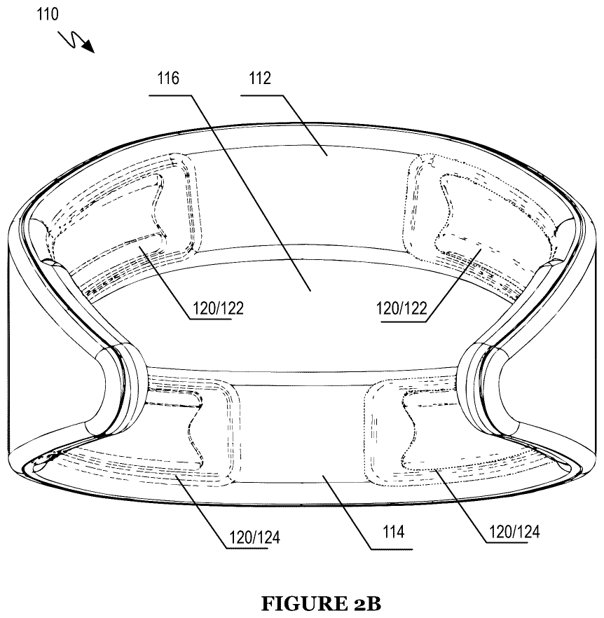

As more specifically shown in FIG. 2A and FIG. 2B, a variation of the system may be designed to provide stimulation across multiple zones. In particular, the system may include a headset frame 110, a plurality of electrodes 120 integrated into the headset frame comprising a first subset of electrodes 122 integrated within the headset frame 110 for a first region and a second subset of electrodes 124 integrated within the headset frame 110 for a second region, and control circuitry 130 electrically coupled to the electrodes. The first subset of electrodes 122 and the second subset of electrodes 124 may each target one or more brain region(s). In one preferred variation, the first subset of electrodes 122 is oriented for a pain region and the second subset of electrodes 124 is oriented for a mood/anxiety region.

The headset frame 110 functions as the structural body of a wearable neurostimulation device. The headset frame 110 may be implemented in a variety of form factors such as a hat, helmet, or a headband. As shown in FIGS. 1-3, one preferred variation may use a headband form factor that partially wrap around a subject's head when worn and be positioned to sit over targeted regions.

The physical design of the headset frame 110 may be designed so as to promote approximate positioning of the device on the head. In some variations, the form of the headset frame 110 may include elements that are intended for easier relative positioning of that element to a fixed part of the body. For example, a headband form factor may include pieces that curve behind the ears in a consistent manner. In another variation, the curvature of the device may be designed to follow typical head curvature to reinforce consistent/reliable positioning. For example, the curvature of the headset frame 110 may have curvature mapping to typical curvature for the back region of the parietal portion possibly to the occipital portion of the skull.

The structural design of the headset frame 110 and its reinforcement of approximate positioning can facilitate easier calibration. As described herein, the system may include alternative calibration mechanisms such as using an accelerometer, gyroscope, tilt sensor, or other positioning system. However, in some variations, the physical positioning of the headset may be the main or only mechanism for calibration.

In one variation, multiple groupings of electrodes are integrated in proximity to targeted regions. In particular, there may be a first subset of electrodes 122 integrated within a portion of the headset frame 110 that generally will be positioned in proximity to a first targeted region (e.g., near regions associated with neuromodulation of pain), and there may be a second subset of electrodes 124 integrated within a portion of the headset frame 110 that generally will be positioned in proximity to a second targeted region (e.g., near regions associated with neuromodulation of mood).

In one variation, the headset frame 110 includes a rounded structure that at least partially conforms to a head curvature profile. In some variations, the headset frame 110 has a headband structure, where there are at least two portions wrap around to opposing sides of the head. In other variations, the headset frame 110 may be a cap that is a concave shape that fits on the head. In one preferred variation, the headset frame 110 may be flexible or be otherwise be designed to be a one-size fits-all (or most) design. However, in some variations, headset frames 110 of different sizes may be used.

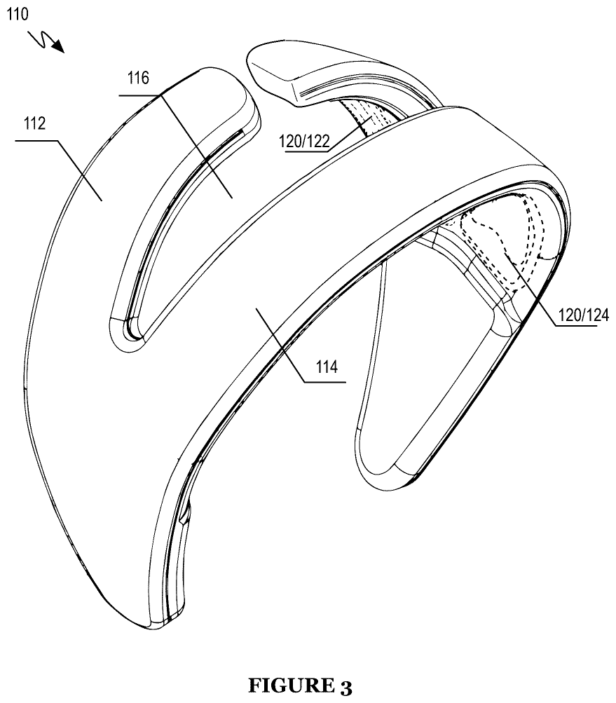

In one variation, the headset frame 110 includes structural features that function to facilitate easy use of the neurostimulation device when there are electrodes positioned for multiple zones. As shown in FIG. 2A and FIG. 2B, the headset frame 110 may preferably include two separated arc structures that join on opposing ends. Both arc structures, in one variation, are continuous. In some variations, at least one of the arc structures may be non-continuous/broken while at least one of the arc structures is continuous as shown in FIG. 3. In this configuration, the headset frame 110 may include an internal opening (i.e., defined through hole) 116. With two arcs connected this way, the headset frame 110 may be a compressive headband frame that can flexibly fit around a variety of head sizes and shapes, but where relative positioning of the two arc structures when placed on the head are in positions approximately consistent with similar brain regions. The dual arc design can enable flexibility in a lateral direction (e.g., for varying head widths). However, the dual arc design substantially restricts change in the angle between the two arc structures, which is helpful in maintaining relative positioning of two stimulation regions.

This dual arc headset frame 110 variation may be used in a neurostimulation device designed for targeting two different brain regions. In this variation, each arc may include groups of electrodes configured for stimulation select regions where that arc would be positioned on the head. Accordingly, the headset frame 110 may include a first arc structure 112 and a second arc structure 114 connected at opposing ends, where the first arc structure 112 curves over a first brain region and the second arc structure 114 curves over a second brain region. A first subset of electrodes 122 may be integrated with the first arc structure 112 and a second subset of electrodes 124 may be integrated with the second arc structure 124. For a dual-arc headset frame 110, the first arc structure and the second arc structure may be arcs that have an angular offset of 15-45 degrees. For example, in one variation, the angle between the two arcs may be 27-30 degrees. In one variation, the angle may be variable. For example, there may be a pivot hinge where the two arcs connect, and the angle may be varied across an angular range, either continuously or through two or more different preset angles.

The electrodes within the headset frame 110 and more specifically individual arc structures (112 and 114) may use 10-20 EEG system. In a 10-20 EEG system, electrode placement may be configured within the headset frame 110 where electrodes are spaced 10%-20% of total front-back or right-left distance of the skull.

In some alternative variations, the headset frame 110 can be implemented as a patch, where it is intended to be adhered, fixtured, or otherwise attached to a particular position on a head of a subject.

In some variations, headset frame 110 may include a plurality of distinct patches, which may require independent attachment to a subject. The separate patches are conductively coupled through an electrical wire or cord. The separate patches may alternatively be conductively coupled through another element. Each patch can include one or more electrodes.

In one variation, a comb electrode design described herein may be used such that one or more sets of electrodes may be integrated into a comb-like frame. A subject could slide one, two or more stimulation combs into their hair as a way of establishing an interface between the electrodes with the skin of the subject.

The plurality of electrodes 120 functions as a selectable array of electrodes that can be used to adjust the targeted region of stimulation.

The plurality of electrodes 120 is preferably integrated into the headset frame at or around a targeted region for neurostimulation. This targeted region may vary depending on the application. For example, a neurostimulation device for pain may have the electrodes integrated into a general region on the headset frame 110 where neurostimulation may be beneficial for addressing symptoms of, or the perception of, pain. In another example, a neurostimulation device for modulating mood may have the electrodes integrated into a general region on the headset frame 110 where neurostimulation may be beneficial for addressing symptoms of mood, such as improving low mood, stabilizing mood, or otherwise.

As discussed, in other variations, the system may include a plurality of electrodes in different sub-regions and/or across a larger region such that two or more distinct “stimulation sites” can be subject to neurostimulation. In this way, different subsets of electrodes may function as distinct electrode groups. These subsets of electrodes (or “electrode groups”) may function to target different brain regions. The targeted brain regions may transcend distinct individual brain regions. Electrical stimulation may be controlled in parallel or independently for the different electrode groups. Herein, the systems and methods are described primarily how it would be applied around one “stimulation site”. Accordingly, the plurality of electrodes 120 may include two subsets of electrodes: a first set of electrodes 122 and at least a second sub of electrodes 124.

In some variations, the electrodes 120 are integrated into the headset frame 110 so as to be positioned when the neurostimulation device is worn to be located around one or more targeted regions. A subset of electrodes may be positioned to target regions associated with mood (e.g., the dorsolateral prefrontal cortex or DLPFC) and/or pain (e.g., the motor cortex or MC). A 10-20 EEG system map overlaying such targeted positioned may be used.

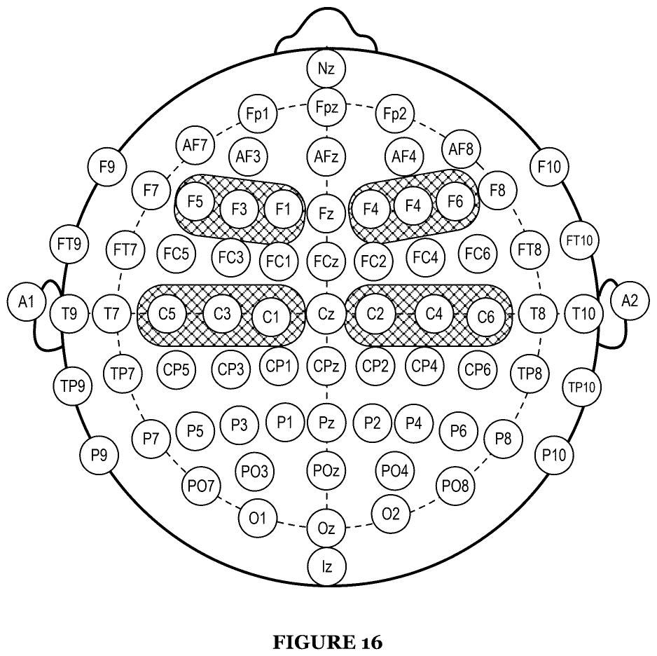

As mentioned, one variation of the neurostimulation device may have two zones that are targeted through two subsets of electrodes. In particular, the two regions may be DLPFC and MC. In one variation, two electrodes may be positioned and integrated into the headset frame 110 to be placed bilaterally over the motor cortex (M1 area, C3-C4 in the EEG 10-20 system), while two other electrodes are designed to be placed bilaterally over the dorsolateral prefrontal cortex (DLPFC, F3-F4 area in the EEG 10-20 system) regions of the brain as shown in FIG. 16.

The M1 may be targeted given its inputs to the posterior insula, which may be associated with perceiving and processing pain sensitivity and may have impaired functional connectivity in pain syndromes. Such regions may be used for targeting chronic pain relief in some instances. There may also be strong connections between the posterior insula and the primary motor area (M1) of the cortex, which may be useful in directing targeting given that the posterior insula is located deep in the subcortical regions of the brain presenting potential challenges in stimulating in a non-invasive manner directly.

The DLPFC may be targeted as being a node for processing interpretation of low mood and anxiety symptoms from the limbic system. Targeting the DLPFC may function to help in reducing depression and anxiety symptoms in some instances. The electrodes may be used in supplying transcranial direct current stimulation (tDCS) to non-invasively engage such mechanisms of the DLPFC with a safe, possibly at-home option.

The neurostimulation device may be configured to use a 4-point montage where there are at least four electrodes placed for contact with the body/scalp of a subject. In such a 4-point montage there may be at least two electrodes placed for stimulation of a first region and two electrodes place for a second region. A montage may include electrodes on both the left and right side. As discussed herein, in some cases, multiple electrodes may be used to “steer” or dynamically adjust exact placement of stimulation to account for different factors.

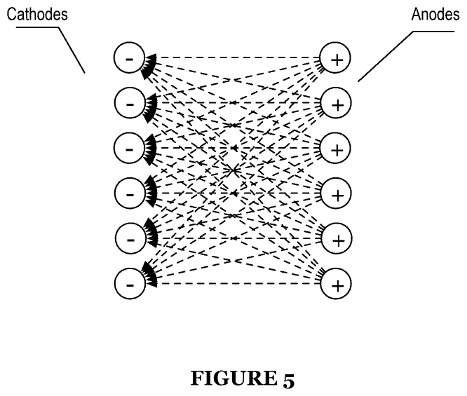

In one variation, within the plurality of electrodes 120 there may be an array of anode electrodes and an array of cathode electrodes. In this variation, anodes and cathodes may be selectable individually or in subgroups as shown in FIG. 5. Selection of anode and/or cathode electrode selection may be used to manage steering and/or positioning of neurostimulation.

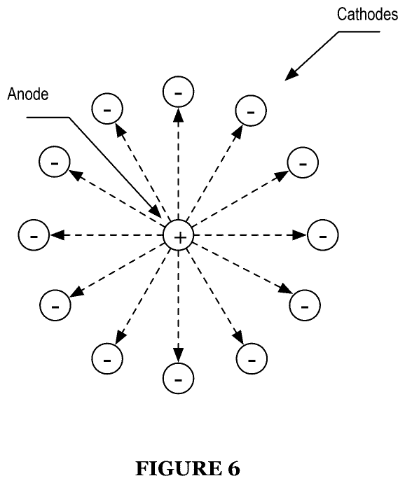

Alternatively, in some variations, the plurality of electrodes may include either a single anode or cathode electrode. For example, the plurality of electrodes 120 may include a single anode electrode and an array of cathode electrodes as shown in exemplary FIG. 6. In another example, the plurality of electrodes 120 may include an array of anode electrodes and a single cathode electrode.

In another variation, one or more electrodes may have controlled and changeable polarity such that an electrode may be activated as an anode or a cathode.

The set of electrodes can be arranged in a variety of formations depending on application. In one variation, a set of electrodes of the same polarity (or different) can be arranged along a one-dimensional path. In one implementation, an array of electrodes may be arranged along a linear path. The path could alternatively be curved, a non-continuous path, or have any suitable arrangement. Anode and cathode electrodes may have similar or mirrored arrangements as shown in FIG. 5, but they could alternatively have different arrangements.

In another variation, a set of electrodes of the same polarity (or different) could be arranged in a two-dimensional array, where the electrodes are dispersed over a region or surface.

In some variations, a set of electrodes of the same polarity (or different) may be arranged in a non-regular pattern. For example, the electrodes may be configured without a regular pattern and positioned based on some external factor.

The spacing of electrodes within an array can impact the resolution and precision for which stimulation position is controlled.

As shown in FIG. 5, an array of anode electrodes may be positioned along one path and an array of cathode electrodes may be positioned along a second path. This may be used to enable selectable effective stimulation at different positions between the two sets of electrodes. For example, the stimulation could be shifted linearly up and down along the paths. In another example, the stimulation may also be angled in different directions.

As shown in FIG. 6, an alternative arrangement of electrodes may include a centrally located anode electrode with a ring of cathode electrodes positioned around the anode electrode. This may be used so that the radial position of stimulation could be moved about the location of the anode. For example, this may be used for HD-tDCS (high-definition transcranial direct current stimulation), tACS (transcranial alternating current stimulation) or temporally interfering electric fields.

An electrode of the set of electrodes may be configured to have circuitry control of the binary state of the electrode. For example, an electrode may be switchable between an active and inactive state. In another variation, an electrode could include discrete controllable states between different polarities. For example, an electrode may be switchable between an active anode state, active cathode state, and a deactivated state.

In another variation, an electrode of the set of electrodes may be configured to specific electric potential to control stimulation magnitude (e.g., voltage and/or current). In some variations, this can be used to control the strength of stimulation. In some variations, this may additionally or alternatively be used to approximate a simulated electrode position based on the superposition of stimulation magnitude of multiple electrodes. For example, two adjacent cathodes may be activated at 50% intended magnitude to simulate a cathode positioned between the two adjacent cathodes. The relative magnitude can be adjusted to modulate the simulated position of a “virtual electrode” to compose the field in a desired location.

The electrodes may use a variety of electrode designs for establishing conductive contact between the electrode and the skin of the subject. Different variations of electrodes may use different materials and/or form factors (i.e., shapes). In some exemplary implementations, the electrodes may be squares or circles made from hydrogels, cellulose sponge, or other moisture preserving material, or Ag/AgCL.

In some variations, an electrode or a group of electrodes may be exposed to or connect to an electrode pad. An electrode pad in one preferred variation may be a sponge that may be wetted for use, a gel pad, adhesive electrode pad, a conductive rubber pad, and/or any suitable component to facilitate conductive contact between electrical contacts of an electrode site and body of a subject wearing the neurostimulation device.

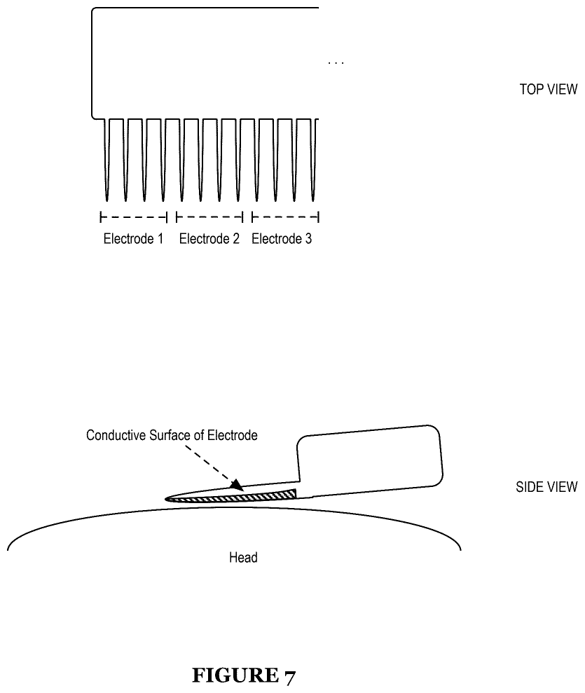

In one variation, the electrodes may be an enhanced electrode design that is a comb toothed electrode as shown in FIG. 7. A comb toothed electrode functions to increase the surface area of contact between the electrode surface and skin for more comfortable stimulation. A comb tooth electrode is an electrode element that includes an extended structure (i.e., an electrode tooth extended along some defined “length”) with at least one conductive contact surface along the defined length of a tooth. The comb toothed electrode will generally include a collection of teeth grouped in proximity. The separate teeth may be of different or the same length as each other.

A tooth could be a flexible, semi-rigid, or even a rigid extension. In one variation, the tooth is thin extended element with a more cylindrical and longitudinal form. In another variation, the tooth could extend along a defined length but include a wider or flatter surface on at least one side. The wider or flatter surface may function to increase surface area where conductive contact can be made with skin. The conductive surface of the tooth could be a conductive pad or other surface. Alternatively, the tooth may be made of a conductive material.

In one preferred variation, the enhanced electrode design can have one or more electrodes in a comb tooth electrode configuration such that the device (the “comb”) can be slid into place on the subject's head. The tangential placement of the device functions to use the mechanical properties of the comb electrode to promote hair being pushed out of the way of the electrically conductive elements. The teeth will generally be curved to conform to the head. Curvature and design of the teeth may be configured specifically depending on where on the head the device is to be placed.

A series of teeth can be integrated into the device. In one variation, subsections of teeth could be used as unified electrode. Multiple different subsections of teeth may be used to establish an array of electrodes. For example, a comb including 30 teeth may have groups of five adjacent teeth conductively coupled so that there are six controllable electrodes. In some cases, there may also be non-electrode teeth included in the comb structure. In another variation, an array of electrodes could have individually controlled teeth electrodes where a single electrode is a single tooth.

The control circuitry 130 functions to electrically control and manage the neurostimulation. The control circuitry can be electrically coupled to each electrode. The control circuitry 130 may include switching or stimulation circuitry such that the control circuitry 130 can control stimulation state of the plurality of electrodes. The control circuitry 130 may include a processor and/or a circuit-implemented system for control of the electrodes.

In a neurostimulation device with electrodes to target two zones, the control circuitry 130 may include control circuitry to independently control neurostimulation applied to electrodes for each zone (e.g., the first subset of electrodes 122 and the second subset of electrodes 124).

The control circuitry 130 may be configured to facilitate the calibration, operation, sensing positioning, and/or other updates to the system.

The control circuitry 130 can be used in establishing the stimulation to target different positions. This can include activating and deactivating different subsets of electrodes. The system may use static direct current stimulation, alternating current stimulation, and/or other patterns of stimulation for the selected active electrodes.

The control circuitry 130 may also be used in calibrating positioning of stimulation. This may also be used in adjusting stimulation positioning based on initial calibration or changes in calibration. For example, if the headset frame is detected to have moved relative to the subject, then the control circuitry 130 may dynamically adjust the stimulation state of the electrodes.

The neurostimulation device can additionally include a power source and/or include a power connection to connect external power. In one variation, the neurostimulation device includes a rechargeable battery. In another variation, an external battery pack may connect to the neurostimulation device.

The system may include one or more control user interface 140 components, which function to offer interfaces for interacting with and/or operating the neurostimulation device.

In some variations, the neurostimulation device may be usable as a standalone device without integration with other external devices or applications. The neurostimulation device may include a control user interface 140 with suitable user input/output components such as buttons/switches, display, indicator lights, and/or other electrical components that may be used to control and/or use the neurostimulation device. In some variations, the neurostimulation device may interface with external computing systems, which may provide additional or alternative component for managing control or operation of the device.

The control user interface 140 may include or interface with a management application 142 which functions as a user interface through which input can be received and/or feedback can be delivered. In one preferred variation, the management application 142 may be operable on a personal computing device such as a smart phone or a computer, but any suitable type of computing device may be used. The neurostimulation device may communicate with a management application 142 using Bluetooth though any suitable wireless or wired connection may be used.

In one variation, the management application 142 may manage stimulation session scheduling. The management application 142 may include configuration to determine an appropriate schedule, adjusting or updating the schedule based on changing conditions (e.g., missed or extra stimulation sessions or based on subject feedback). In one example, the management application 142 may access or interface with health data on menstrual cycle patterns of a subject and then plan stimulation sessions according to predicted patterns. Alerts or notifications may then be triggered within the system (e.g., on the neurostimulation device or within an application on a computing device) to prompt following of a stimulation schedule. The usage and adherence to the stimulation session schedule may be tracked based on usage of the neurostimulation device and then used to automatically update the stimulation session schedule which may impact altering scheduling, altering future stimulation session parameters (e.g., duration, stimulation intensity, stimulation patterns, and/or distribution across different regions).

In another variation, the management application 142 can facilitate directing a subject through a position calibration process. Different processes may be implemented through the management application 142 to facilitate position calibration. For example, a positioning system (e.g., using an accelerometer or gyroscope) and/or some orientation indicator (e.g., to signal right or left side of the device).

In another variation, the management application 142 may facilitate testing for conductive contact by applying stimulation and testing of a conductive path is achieved. This condition may be verified based on achieving some impedance threshold (having impedance below the threshold) and then indicating when proper positioning is achieved. In some variations, the management application 142 may generate instructions during placement to provide feedback to a subject until the neurostimulation device has a suitable position.

In one such variation, the management application 142 may generate positioning feedback indicating proximity to satisfying a positioning condition. The positioning condition may be based on various conditions. One positioning condition is based on achieving a conductive contact for stimulation using the plurality of electrodes 120.

Another positioning condition may be based on sensed orientation of the device. A position sensing system 150 may be used to detect if the neurostimulation device satisfies some positioning condition. In one such variation, the position sensing system 150 may detect if the device has an approximately correct orientation relative to a gravitational direction. In a dual arc variation of a neurostimulation device, the position sensing system 150 may use an accelerometer or other suitable sensing system to detect if the appropriate arc is more aligned at the top of the head. This may be used to confirm the device is not worn backwards.

As another related positioning condition, sensors may be used to detect physical contact or pressure on the underside of the neurostimulation device. For example, a moisture sensor may be used when sponges are used as electrode pads, where the amount of moisture sensed may be associated with an amount of pressure of device onto the head.

In one variation, the management application 142 may include configuration to output positioning calibration feedback. This may be a visual/or audio feedback. In one variation, the positioning calibration feedback may indicate or relate to proximity of satisfying one or more positioning conditions. In one particular variation, random or pseudo random noise may be applied to the positioning calibration feedback. To the user, they would see an indicator showing varying closeness to getting the device properly positioned, while they are presumably attempting to properly place the device. This noise added to the feedback may function to prompt or encourage the subject to continue altering positioning of the neurostimulation device. Test stimulation signals may be continuously or periodically performed when generating this positioning calibration feedback so that when appropriate conductive contact is made the feedback can change to indicate they should stop or that they are closer to getting it properly positioned. If conductive contact is achieved, then the positioning calibration feedback can update. It may update to indicate the device has position properly calibrated. In some cases, a confirmation test may be performed to make sure conductive contact is maintained after the subject stops adjusting position. In another variation, the positioning calibration feedback may be a time weighted average of if the device has conductive contact. In this way, if the device temporarily achieves proper conductive contact, the feedback may indicate that the user is closer to properly positioning the device, and if the device is kept in a position with proper conductive contact for the full length of the averaged time window, then the feedback will indicate position has been calibrated.

In one preferred variation, the management application 142 can output instructions on positioning the neurostimulation device. Depending on position of the neurostimulation device, a physical sensation resulting from a test stimulation (e.g., phantom tingling or vibration sensation) can be used to predict/determine position of the device and then perform dynamically positioned stimulation based on this reference point. After a test stimulation, the management application may be used in collected feedback from the subject on any sensations from the stimulation. For example, a subject may indicate if and where and when any tingling sensation is felt. When felt, this may give the system information on the position of the neurostimulation device relative to the head of the subject.

More specifically, the management application 142 can be used to perform some stimulation test, receive input, and based on the input predict location and/or stimulate in a different position. This version may be useful when multiple dynamic electrodes are used so that the exact configuration of electrodes can be determined. In some cases, a new test stimulation position can be performed using the dynamic electrodes. In some cases, the stimulation position may be changed by directing the subject to move the device.

In a related variation, the neurostimulation device may include audio, visual, and/or a haptic feedback element that are triggered during an initialization or calibration stage. The feedback elements on the device may be used to indicate direction of the device. For example, haptic feedback may be triggered on the right side of the device to help the user confirm the device is correctly pointed in the right direction.

The management application may also be used to control the neurostimulation device. The management application 142 may also be used for receiving and presenting information from the device or facilitating other forms of user interaction. In some variations, the management application 142 may be used for collecting user input as feedback. For example, the management application 142 may collect user input on results after a stimulation session. For example, user input may collect feedback input on a subject's mood and/or pain before and/or after stimulation sessions. This may be used to adjust subjective stimulation sessions.

In some variations, the system can include a position sensing system 150, which functions to detect position and/or movement of the neurostimulation device.

In one variation, the position sensing system 150 can include an inertial measurement unit or other sensor system that may include or be a digital accelerometer, a gyroscope, and/or a magnetometer. Additionally or alternatively, the position sensing system 150 may include other sensing mechanisms such as a tilt sensor or other mechanisms to detect motion and/or orientation/position. In particular, an accelerometer integrated into the headset frame may provide information on the general orientation of the headset frame. The angular position extracted from the accelerometer data can indicate some information used to predict how the headset frame may be positioned on an individual (assuming a head being in a standardized position such as having the head positioned in an upward position).

When used to calibrate position, the positioning sensing system (e.g., an accelerometer) may be configured to monitor movement patterns when a user puts on the headset frame to enhance predictions of device position. For example, the movement data stream may be used to detect a placement signature pattern that is indicative for how the device is put onto the subject. A placement signature that matches (or has high correspondence to) a calibrated placement signature can be used to predict that the system is likely in a calibrated position.

The accelerometer, inertial measurement unit, or more generally the position sensing system may also be configured to detect movement of device after placement calibration. This may be used to dynamically adjust stimulation to adjust for small movements of the device.

In some variations, the system may include a second reference movement positioning system from which to base predictions. For example, inertial or movement data from another device, such as a smart phone, smart earphones, or smart eyeglasses, may be used to differentiate between movement of the head or body from movement of the neurostimulation device relative to the head.

### 3. Method

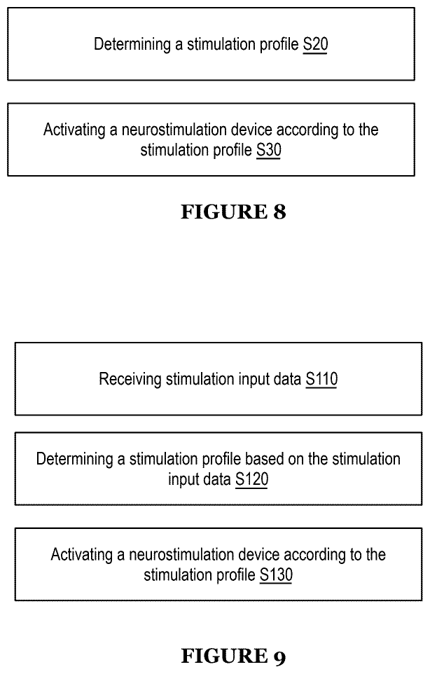

A method for enhanced neurostimulation may include determining a stimulation profile S20; and activating a neurostimulation device according to the stimulation profile S30 as shown in FIG. 8. A stimulation profile may be personalized and adapted to impact use of a neurostimulation device. The stimulation profile may be configuration determining how stimulation is applied (e.g., the stimulation of the electrodes) to how stimulation sessions are administered (e.g., scheduling of sessions).

The method will generally use one or more data inputs to determine the stimulation profile. As shown in FIG. 9, the method may more particularly include receiving stimulation input data S110; determining a stimulation profile based on the stimulation input data S120; and activating a neurostimulation device according to the stimulation profile S130.

This method may use various inputs to customize stimulation of the neurostimulation device. In some variations, the method may be employed within neurostimulation systems intended for use directly by a subject without direct assistance from a trained professional. Different inputs may be used to enable the neurostimulation device to be more easily operated by a subject when self-administering neuromodulation. One such use case would be for a neurostimulation system intended for regular use by women for treatment of menstrual cycle symptoms such as pain, mood affliction, or cognitive impairment.

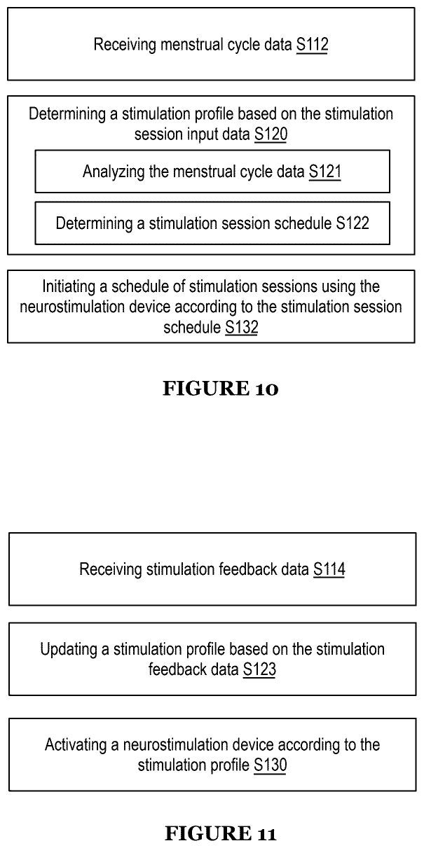

In some variations, such as shown in FIG. 10, the method may automate planning and administering an appropriate schedule of stimulation sessions (e.g., based on menstrual cycle data).

In some variations, such as shown in FIG. 11, the method may facilitate personalization and dynamic adjustments based on different forms of feedback or detected changes, which may be used for improving results and automatically enabling enhanced operation of a neurostimulation system.

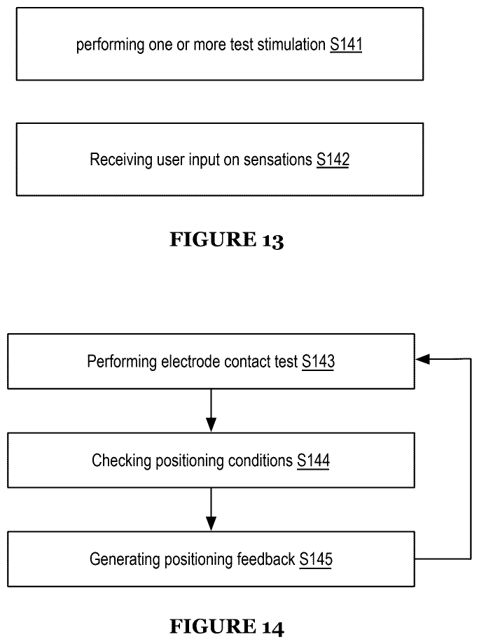

In some variations, such as shown in FIGS. 13, 14, and 15, the method may facilitate calibrating positioning of the neurostimulation device. When using a neurostimulation device with two zones of stimulation, which may be particularly helpful in neuromodulation treatments for women's health like for menstrual cycle symptoms, calibration may facilitate aligning or targeting stimulation across multiple targeted regions of simulation. When using a neurostimulation device with an array of electrodes, stimulation may be targeted at intended locations of a subject as in the variation in FIG. 16.

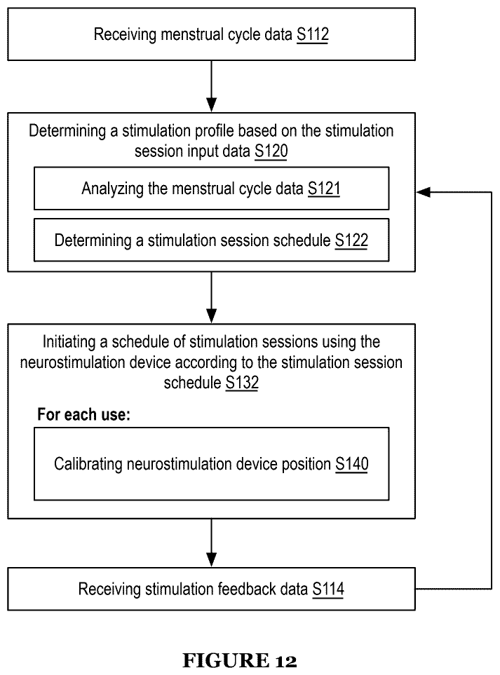

Many of these variations may be used in any suitable combination. In one exemplary variation shown in FIG. 12, a method variation may automate planning and administering an appropriate schedule of stimulation sessions (e.g., based on menstrual cycle data), calibrate positioning of the neurostimulation device—potentially aligning electrodes for stimulation across two zones, and/or personalize neurostimulation applications based on different forms of feedback or detected changes for improving results for subsequent stimulation sessions.

The methods described herein may be used in combination with a system such as described above. However, some variations of the methods may be used with other suitable neurostimulation systems.

Block S110, which includes receiving stimulation input data, functions to use one or more inputs to alter use of a neurostimulation system. In some variations, the input data may be input received from a user through a user interface. The user interface may be a graphical user interface operable within a management application. The user interface could alternatively be one provided on the neurostimulation device. The input data could include data from an outside system or service. For example, health data from a third-party service may be used. The input data may also be sensor data or other state data from the neurostimulation device.

Block S120, which includes determining a stimulation profile based on the stimulation input data, functions to determine various aspects of how neurostimulation should be administered. The stimulation profile can define one or more parameters related to stimulation sessions and/or applied stimulation during a session. The stimulation profile may include a stimulation session schedule that characterized recommended or permitted times or conditions for stimulation sessions. The stimulation profile may additionally or alternatively include stimulation configuration parameters that define at least one aspect of how stimulation is applied during a session. Stimulation configuration parameters may define intensity (e.g., current amount), stimulation patterns (e.g., frequency or stimulation signal patterns), duration of a stimulation session, the electrodes used within an array of electrodes, balance of stimulation across different regions (if there are multiple regions of stimulation) and/or other aspects.

Block S130, which includes activating a neurostimulation device according to the stimulation profile, functions to trigger operation of the neurostimulation device.

In some variations, activating the neurostimulation device may include prompting or managing use. This may be used in managing stimulation sessions. In such a variation, activating the neurostimulation device may include generating alerts or notifications, which may be communicated through a management application (e.g., a user application operable on a computing device) or through indicators on the device.

In some variations, activating the neurostimulation device may include activating stimulation of electrodes of the neurostimulation device. This can include driving current through at least two electrodes. In one preferred variation, a stimulation session may include applying current with 2 mA and for 20 minutes. However, other montage variations may alternatively be used for the stimulation configuration.

In some variations, the neurostimulation device comprises a set of electrodes integrated into two distinct stimulation regions of the neurostimulation device. The neurostimulation device may include a first subset of electrodes integrated within a headset frame (or body) of the neurostimulation device with a shape conforming to a first stimulation region of a defined head shape, and a second subset of electrodes integrated within the headset frame of the neurostimulation device with a shape conforming to a second stimulation region of the defined head shape. Activating the neurostimulation device may simultaneously stimulate across both regions. In some variations, activating the neurostimulation device may apply stimulation across the two neurostimulation in alternating or varying ways. In some instances, the amount and/or form of stimulation (e.g., duration, intensity, pattern of stimulation) may be independently varied or weighted across the two regions. The stimulation profile may define different ways in which stimulation is applied, distributed, and/or weighted/varied across the two regions. As detailed below, applying stimulation across two electrodes may be based on different input data.

In some variations, the method for using stimulation input data to determine a stimulation profile may be adapted for determining a stimulation session schedule based on various health data inputs. This method variation may be particularly applicable in using neuromodulation for treatment of menstrual cycle symptoms.

In this variation, receiving stimulation input data S110 may include receiving menstrual cycle data through a computer interface S112; determining the stimulation profile based on the stimulation input data S120 may include analyzing the menstrual cycle data S121 and determining a stimulation session schedule S122; and activating the neurostimulation device according to the stimulation profile S130 comprises initiating a schedule of stimulation sessions with the neurostimulation device according to the stimulation session schedule S132.

Accordingly, as shown in FIG. 10, the method may include receiving menstrual cycle data through a computer interface S112; determining the stimulation profile based on the stimulation input data S120, which comprises analyzing the menstrual cycle data S121 and determining a stimulation session schedule S122; and initiating a schedule of stimulation sessions with the neurostimulation device according to the stimulation session schedule S132. This method may additionally be adjusted for scheduling stimulation sessions relative to other predictable and/or cyclical health events.

Block S112, which includes receiving menstrual cycle data through a computer interface, functions to collect data that may be used to predict or indicate menstrual cycle timing.

In one variation, receiving the menstrual cycle data through a computer interface is received through user input within a user application. In this variation, a subject may use a user application such as the management application described above to supply input used to track menstrual cycles. This may include indication of a start date and end dates of their menstrual period. The management application may additionally collect additional health related data which may be useful in scheduling stimulation sessions and/or otherwise augmenting stimulation profile configuration.

The management application may additionally collect input related to symptoms such as cramps/pain, mood, breast tenderness, bloating, headaches, or other symptoms. The type and severity of these symptoms as well as optionally start date and/or end dates of the symptoms may be tracked.

The management application may additionally collect additional information such as birth control use and/or medicine use. Other activity may additionally be tracked such as sleep. For example, the management application may additionally collect informational data on sleep and/or exercise.

In some variations, receiving the menstrual cycle data through a computer interface is received through a data interface to a third-party health data service. The management application or some data services with data interface may access data collected from other device or digital service. The third-party health service may be a health data framework provided through an operating system. The third-party service may be a health data service accessed via a web application programming interface (API). The third-party health service may also be health data collected from a health or wellness computing device such as a smart wearable computing device.

In some variations, some health data may be collected through user input into a user interface and some health data may be retrieved from a third-party health data service.

Blocks S121 and S122, which include analyzing the menstrual cycle data S121 and determining a stimulation session schedule S122, function to use health data to plan for a next menstrual cycle. The systems and methods herein may use neuromodulation before, during, and/or after periods of a subject's menstrual cycle. Neurostimulation may provide particular benefits to a subject prophylactically and so stimulation sessions may in some instances may be administered multiple times before a period starts. Accordingly, the method and blocks S121 and S122 may generate a stimulation session schedule that sets a schedule of multiple stimulation sessions in advance of a predicted start of a period. The stimulation session schedule can function as a stimulation treatment plan which is based on menstrual cycle data.

Analyzing the menstrual cycle data S121 and determining a stimulation session schedule S122 may include predicting the next menstrual cycle and scheduling a sequence of stimulation sessions for multiple days preceding a start of a next period in the next menstrual cycle. This may include scheduling a sequence for different days before, during and/or after a period. In one variation, the stimulation session schedule may schedule stimulation sessions during the luteal phase (e.g., during part or the whole phase). Scheduled sessions may be added or removed based on various outcomes or events. Additionally, the stimulation configuration (how stimulation is applied) may be adjusted for different portions of the schedule. For example, around ovulation, stimulation session schedule may include setting stimulation configuration for use of a montage focused on a pain region only (e.g., not a mood region). For a dual-zone neurostimulation device, this may result in a subset of stimulation sessions to only use one of the zones depending on when a session is scheduled within the overall schedule.

In one variation, determining the stimulation session schedule may include scheduling 5-6 sessions of neurostimulation device use. These may include stimulation sessions before a predicted start of a period, during, and/or after the period. Sessions may be scheduled for each day, but in some cases, there may be multiple sessions in a single day. In one example, the stimulation session schedule may start five days prior to a predicted start of a period, be used during the period and then 1-2 days after the period ends. The duration of those sessions may also vary depending on the schedule. Determining the stimulation session schedule may additionally identify or plan optimal times of a stimulation session and/or planned settings of stimulation sessions. In some variations, different stimulation sessions may have different stimulation configuration parameters.

As discussed, the neurostimulation device may include a set of electrodes integrated in two (or more) distinct stimulation regions of the neurostimulation device. The stimulation sessions that are scheduled may stimulation sessions with planned neurostimulation of the two distinct regions (two specific zones associated with different forms of neuromodulation). In some variations, this may include applying neurostimulation across two regions during each session to address pain and anxiety associated with the menstrual cycle during the scheduled sessions.

Neurostimulation across the two regions may be schedule dependent. Accordingly, in some variations, determining the stimulation profile may include determining the stimulation session schedule, but may additionally include setting stimulation configuration for balancing stimulation across the two distinct stimulation regions. This may include setting stimulation configuration (e.g., montages) for different subsets of sessions in a stimulation session schedule. For example, the weighting or balance of stimulation across a first region (e.g., associated with mood) and a second region (e.g., associated with pain) may vary depending on when the session falls within the schedule.

The stimulation session schedule will generally use the predicted timing of a subject's period as one data input. Other input data may also be used. For example, indication of a subject's main symptoms, patterns in occurrence of those symptoms, feedback on previous sessions, patterns in use of the neurostimulation device, adherence to stimulation session schedule, and/or other collected data may be used to personalize and customize the stimulation session schedule.

As described below, collected feedback data (S114) may additionally be used to adjust a stimulation session schedule and/or a stimulation configuration/montages for different sessions even in the middle of a treatment plan.

Block S132, which includes initiating a schedule of stimulation sessions with the neurostimulation device according to the stimulation session schedule, functions to administer or direct usage for the stimulation session schedule.

As one aspect, initiating the schedule of stimulation sessions may include notifying or communicating to a subject the stimulation session schedule. In one variation, initiating a schedule of stimulation sessions with the neurostimulation device may include triggering a session reminder through a user interface. Communicating the stimulation session schedule may involve triggering alerts on a computing device (e.g., the management application or on the neurostimulation device), scheduling use on a digital calendar, or otherwise prompting a subject on when to use the neurostimulation device.

The user interface may be through the management application. For example, the management application may include a user interface displaying the stimulation session schedule. In another example, the management application (or a related web service) may trigger push notifications, text notifications, email reminders and/or other digital communications.

The user interface used to communicate information about the schedule may additionally or alternatively be provided through the neurostimulation device directly. For example, a neurostimulation device may include a session indicator. The session indicator could be a light or an audio signal. The session indicator may activate when it's time for a subject to use a session. The indicator may be deactivated or turned off during time windows when a user is not planned to have a stimulation session.

In one variation, initiating the schedule of stimulation sessions may additionally include setting and/or enforcing usage windows of the stimulation session schedule. This may include enabling or disabling use of the neurostimulation device according to the schedule. This may function to restrict usage so that it may not be overused by a subject. This may include enabling use of the neurostimulation device during a time window associated with a scheduled stimulation session. This may also include disabling or limiting use of the neurostimulation device outside of a time window associated with a scheduled stimulation session and/or after completion of a scheduled stimulation session.

Initiating a schedule of stimulation sessions with the neurostimulation device according to the stimulation session schedule may also include activating neurostimulation of the neurostimulation device. As described above, in some variations, different stimulation configuration may be set for different sessions or groups of sessions. Accordingly, activating neurostimulation may include using neurostimulation configuration associated with each session.

As mentioned, the use of a neurostimulation device that targets two zones, namely a pain region and a mood region, may have particular benefits for treating symptoms related to menstruation. Accordingly, the neurostimulation device may include a set of electrodes integrated in two distinct stimulation regions of the neurostimulation device. Activating the neurostimulation device according to a stimulation profile may include during the stimulation sessions activating stimulation across at least two regions (e.g., a pain related region and a mood related region). In some variations, neurostimulation across two or more regions may be customized for each region. This may additionally be varied for different sessions in the stimulation session schedule.

In some variations, the method may collect feedback data through S114 that relates to usage of the stimulation device. This may be used to track adherence to the stimulation session schedule and updating accordingly depending on how a subject follows the stimulation session schedule. Accordingly, the method may include tracking usage of the stimulation device relative to the stimulation session schedule and updating the stimulation session schedule based on the usage. In the event of a session being missed, subsequent sessions may be adjusted. This may include adding more sessions. For example, if the session for one day is missed, then the stimulation session schedule may add a second session to the next day with a first session scheduled for the morning and one in the evening. In another example, if a session is missed, then one or more sessions may have their duration extended or other stimulation configuration parameters adjusted to compensate for the missed session.

In the event of a user initiating an additional or unscheduled stimulation session, then the stimulation session schedule may similarly be updated. The updates may reduce the number, duration, or other stimulation configuration parameters based on the additional or unscheduled stimulation session. In one variation, the sessions may be scheduled to follow different conditions or constraints. For example, one constraint may restrict scheduled sessions to no more than 7 days in a row. Another constraint or target may be for there to be no gaps in the schedule more than 2 days in a row. Another constraint may be that there be no more than some maximum number of sessions in a given day (or some other time period) and/or there being a minimum time between scheduled stimulation sessions. In this way, sessions may be added to the stimulation session schedule up to the maximum number of sessions and which satisfy other conditions/constraints.

As mentioned, in some variations, the method for using stimulation input data may use one or more forms of feedback data to update a stimulation profile. This use of feedback data may be used in connection with the method for scheduling stimulation sessions relative to a menstrual cycle (or other predictable health events).

In this variation, receiving stimulation input data S110 may include receiving stimulation feedback data S114; and determining the stimulation profiled based on the stimulation input data S120 may include updating the stimulation profile based on the stimulation feedback data S123. Accordingly, as shown in FIG. 11, one method variation for dynamically adjusting neurostimulation based on feedback data may include receiving stimulation feedback data S114; updating a stimulation profile based on the stimulation feedback data S123; and activating the neurostimulation device according to the stimulation profile S130. These processes may be used in combination with a method for scheduling stimulation sessions as shown in FIG. 12.

In some variations, feedback data may be collected and then used to set or adjust the stimulation profile for subsequent sessions. This functions to set stimulation configuration for a subsequent session. This may additionally or alternatively function to adjust a stimulation session schedule.

In one example, a user may supply feedback on mood or pain after a stimulation session. Their response may be used to adjust how stimulation session. For example, if a user indicates more issues with pain, then neurostimulation may be weighted more greatly on pain related regions when using a dual zone neurostimulation device.

In another example, user feedback may be usage historical data collected from a neurostimulation device. As described above, the method may include tracking usage of the stimulation device relative to the stimulation session schedule and updating the stimulation session schedule based on the usage.

In some variations, feedback data may be collected during an active stimulation session and then used to alter stimulation within the active stimulation session. Accordingly, the method may include receiving stimulation feedback data during an active stimulation session; updating a stimulation profile based on the stimulation feedback data; and updating the neurostimulation device according to the updated stimulation profile.

Block S114, which includes receiving stimulation feedback data, functions to collect input that may be used to signal if changes would benefit operation of the neurostimulation device.

Feedback data may be received through different channels. In some variations, the feedback data may be collected through user input such as supplied through a user interface of the management application.

In some variations, the feedback data may be collected from operation or sensor data from the neurostimulation device. For example, usage data of the neurostimulation device may be used as feedback data. Sensor data such as data from a positioning system may be used to understand positioning or movement of the neurostimulation device.

In some variations, feedback data may be collected from connected devices or third-party data services.

In some variations, the method may include processes to be responsive to feedback data related to biology changes, changes in behavior, and/or symptomatology.

In one variation, receiving stimulation feedback data may relate to updates on biology or health state of a subject. Changes in age, medication, medical events, exercise, sleep, and/or other biological aspects may be used as signals to alter a stimulation profile. In such variations, receiving stimulation feedback data may include detecting or receiving input of a change in biology (e.g., age, medication, etc.). For example, receiving an update on a change in contraception usage may be used to predict change in cycle so that a stimulation session schedule may be adjusted for this change.

In another variation, receiving stimulation feedback data may relate to behavior. In particular, the feedback data may be associated with behavior related to use of the neurostimulation device. The stimulation feedback data may include usage data indicating when and how a neurostimulation device was used. When there is a stimulation session schedule, the usage feedback data may indicate if a session was undergone during the scheduled time window. The usage feedback data may also indicate if the duration of the session followed the schedule (e.g., completing full session, stopping early, or having a longer than planned session). Such usage feedback data may be used to track and detect non-adherence to the stimulation session schedule. The usage feedback data may also be used to understand patterns in use. For example, a particular subject may have behavior patterns indicating preference for shorter stimulation sessions or for conducting stimulation sessions at particular times of day.

In another variation, receiving stimulation feedback data may relate to experienced symptoms. Symptom-related feedback data may relate to mood, pain, cognitive abilities (e.g., levels of focus), sleep, or other symptoms. These may be used to adjust a stimulation profile so that results are enhanced and personalized for the subject.

The symptoms may also be used to adjust steering of stimulation in the variation where an array of electrodes in the neurostimulation device enables stimulation steering. This may be used to dynamically target stimulation to regions of a subject to enhance neuromodulation. The neurostimulation device may sit on different subjects' heads in different ways even when position is calibrated. An array of electrodes may allow stimulation to be dynamically steered to target different locations of stimulation. Over multiple sessions of use, the steering may be adjusted until configuration for electrode steering is personalized for enhanced neuromodulation results.

Block S123, which includes updating a stimulation profile based on the stimulation feedback data, functions to adjust stimulation configuration or schedule based on new feedback data. Block S123 may make the use and operation of a neurostimulation device dynamic to changing conditions.

In one variation, updating the stimulation profile may include adjusting the stimulation session schedule. The schedule may be adjusted to account for changes in biology. In one example, if a period starts earlier or later than anticipated, then the stimulation session schedule may be adjusted. The schedule may be adjusted to account for behavior. As discussed above, in some variations the method may include tracking usage of the stimulation device relative to the stimulation session schedule (as a form of feedback data) and updating the stimulation session schedule based on the usage. If the subject is not adhering to the stimulation session schedule, the schedule session may be adjusted to adapt to those changes. The schedule may additionally or alternatively be adjusted to account for changes in symptomatology. If a user is not seeing significant improvements the schedule may be adjusted to provide more neurostimulation.

In another variation, updating the stimulation profile based on the stimulation feedback data may include adjusting parameters defining electrical stimulation of electrodes in the neurostimulation device. This may be used to adjust how stimulation is applied for at least one session. With a neurostimulation device with dual-zones or multiple zones, this may be used to adjust balancing or variations of stimulation across the different zones. This may also be used to adjust other parameters of a session such as duration, stimulation patterns, stimulation intensity, stimulation steering, and/or other aspects.

In some variations, the method may include or be adapted to calibrate positioning of a neurostimulation device. This may be particularly useful for a neurostimulation device intended for personal use by a subject without supervision or assistance from a trained professional. For a neurostimulation device intended for neuromodulation for menstrual cycle related symptoms, having a device that subject can easily and reliably use at home or within their daily lives is important for making such devices more accessible.

The method variations described herein accordingly may include calibrating neurostimulation device position S140, such as shown in FIG. 12. Calibration can use different stimulation tests while collecting one or more forms of data input to determine if the neurostimulation device is positioned in a way that satisfies different conditions, which may signal proper positioning. Hair styles and head shape of subjects may at times present challenges, which may be addressed by position calibration.

As shown in FIG. 13, in one variation, calibrating neurostimulation device position may include activating a test stimulation S141 and receiving user input on sensations S142. This variation may include applying stimulation in at least one region that produces some physical sensations (e.g., tingling sensations or paresthesia) or results that can be directly reported by a subject. The test stimulation may be activated for an electrode pairing that is associated with a physical result detectable by a subject. In cases where there is an array of electrodes, the test stimulation may cycle through different pairings or stimulation configuration of the electrodes to assist in locating and optionally repositioning the neurostimulation device. The test signal may be repeatedly or continuously be applied while feedback may be provided for a user to move or adjust positioning of the headset until user reports on the sensations.

After a test stimulation, a management application may be used in collecting feedback from the subject on any sensations from the stimulation. For example, a subject may indicate if and when any tingling sensation is felt. When felt, this may give the system information on the position of the neurostimulation device relative to the head of the subject. In some instances, receiving confirmation of a sensation may indicate the neurostimulation device has satisfied a positioning condition. Other conditions may also need to be satisfied before completely calibrated. For example, a suitable number of electrodes may need to have conductive contact with the skin/body of the subject.

Not all regions of the brain may have such immediately detectable physical sensations during neurostimulation. Positioning electrodes used for stimulation of one region resulting in the reported sensation, may be used to assume proper positioning of one or more other electrodes. In some variations, the test stimulation may be used to properly align the neurostimulation device even if the electrodes generating the test signal are not used for subsequent neurostimulation. In other words, the neurostimulation device may include electrodes used for facilitating calibrating positioning.

As shown in FIG. 14, in one variation, calibrating neurostimulation device position S140 may include performing an electrode contact test S143, checking positioning conditions S144, and generating positioning feedback S145. Achieving conductive contact with a subject is one critical condition for properly using a neurostimulation device. This variation may check for conductive contact along with other possible conditions, while also providing positioning feedback. Positioning feedback may be updated to indicate position is calibrated when positioning conditions are achieved.

Block S143, which includes performing the electrode contact test, functions to check repeatedly or continuously if suitable conductive contact is established with electrodes and the body/scalp of a subject. The contact test may activate the electrodes such that a resistance or impedance may be detected. Impedance will generally be high when there is no conductive contact, and then it will drop to a typical level when the electrode has suitable conductive contact.

When there is an array of electrodes, the electrode contact test may cycle through testing different pairings to see if a subset of electrode pairs achieves contact. Depending on the implementation, calibrating position may involve achieving conductive contact for all electrodes or alternatively just a suitable subset of electrodes.

Block S144, which includes checking positioning conditions, functions to evaluate one or more conditions that relate to how a simulation device is positioned. The conditions may generally be based on sensor data, sensed state of the electrodes, and in some cases input from a user.

The positioning conditions preferably includes measuring satisfying a conductive contact condition.

The positioning conditions may additionally include confirming a sensation resulting from a test stimulation as described above. This test may be performed after the conductive contact condition.

The positioning conditions may also include confirming orientation of the neurostimulation device.

In one variation, confirming orientation of the neurostimulation device may include detecting orientation of the neurostimulation device relative to a direction of gravity. This may function to determine if an appropriate portion of the device is oriented upwards. This may be used to make sure a device with a form like in FIGS. 2A and 2B is worn in the right direction on the head.

In another variation confirming orientation of the neurostimulation device may include triggering an output component on the neurostimulation device to signal orientation. The user may confirm (e.g., through the management application) the side the output component was triggered. For example, a haptic feedback element (e.g., a vibrational motor) or an audio speaker may be positioned and activated on one side. The user will know the device is worn with the corrected right/left orientation if the output is experienced on the appropriate side.

In another variation, the positioning conditions may additionally include confirming satisfaction of device pressure conditions. Pressure sensors on an internal side may measure pressure of the neurostimulation device onto a subject's head. The pressure conditions may be satisfied when the pressure is above a pressure threshold. In one implementation, a moisture sensor may be used to approximate pressure based on compression of wetted sponges used as electrode pads.

Block S145, which includes generating positioning feedback, functions to output some form of feedback to the subject to assist in adjusting positioning. In some variations, the positioning feedback may be supplied through the neurostimulation device and/or a management application. The positioning feedback may include visual and/or audio feedback. In one variation, the positioning calibration feedback may indicate or relate to proximity of satisfying one or more positioning conditions. The positioning feedback may be symbolic or parameter based. The positioning feedback may also be descriptive and/or instructional to provide suggestions on how to adjust positioning. This may include providing instructions on directions to move the device and/or adjusting pressure.

In one variation, generating positioning feedback may include augmenting a feedback indicator with a noise signal. This variation may function to use open loop feedback where the feedback indicator encourages continued adjustment of position until conditions are satisfied. The noise signal may be random but may also be based on a defined pattern.

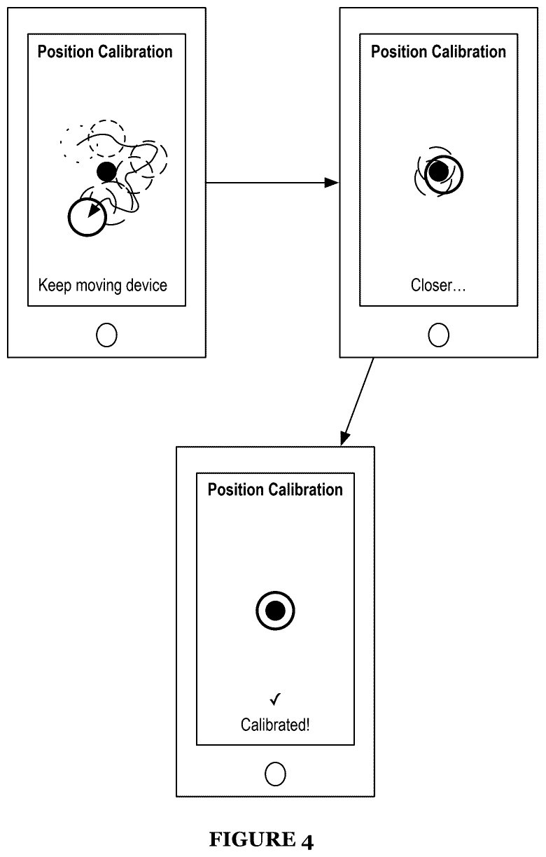

The feedback indicator in one variation, can be a graphical element on a user interface that moves around to different positions with some visual indication when position calibration is close and/or achieved. In the example of FIG. 4, when the open circle is centered around the solid circle, then calibration is achieved.

The feedback indicator may additionally or alternatively be based at least in part on the positioning system and used to encourage the subject to move the device through a range of positions.

When appropriate conductive contact is made (and/or other positioning conditions are satisfied) the feedback indicator can change to indicate they should stop or that they are closer to getting it properly positioned as shown in FIG. 4.

In some variations, calibrating position may be adapted for setting stimulation steering. This may be used in particular when there is at least one array of electrodes. In some variations, there may be multiple arrays of electrodes. The different arrays of electrodes may be used for stimulation different regions. Activation of the array of electrodes may be selectively done to steer stimulation to targeted regions. Calibration of position may be used to determine how to steer electrodes. Feedback input may additionally or alternatively be used to determine a targeted stimulation region.

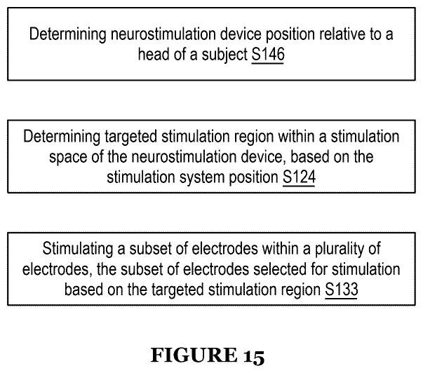

In a variation where steering of electrodes is determined based at least in part on calibration, calibrating neurostimulation device position S140 may include determining neurostimulation device position relative to a head of a subject S146; determining the stimulation profiled based on the stimulation input data S120 may include determining targeted stimulation region within a stimulation space of the stimulation system, based on the stimulation system position S124; and activating the neurostimulation device according to the stimulation profile S130 may include stimulating a subset of electrodes within a plurality of electrodes, the subset of electrodes selected for stimulation based on the targeted stimulation region S133. Accordingly, as shown in FIG. 15, a method for calibrating dynamically positioned neurostimulation can include determining stimulation system position relative to a head of a subject S146, determining targeted stimulation region within a stimulation space of the stimulation system, based on the stimulation system position S124; and stimulating a subset of electrodes within a plurality of electrodes, the subset of electrodes selected for stimulation based on the targeted stimulation region S133.

Block S146, which includes determining stimulation system position relative to a head of a subject, functions to measure or predict position of a neurostimulation device relative to the intended region of stimulation.

Determining the stimulation system position can be performed as an initial calibration process. This positioning process can have relaxed precision requirements since the electrodes may be used to more precisely locate stimulation as long as the electrodes are positioned so that the targeted region is within a region of possible stimulation for the electrodes.

In some cases, such calibration may involve a calibration mode and/or involvement by the subject to achieve calibrated position. Calibrating location can involve calibrating position and orientation of the stimulation system relative to a 10-20 EEG system. This may enable more standardized stimulation mapping to be performed to target specific regions.

In some variations, other position calibration processes may initially be performed, which may function establish a suitable positioning of the neurostimulation device.

In some variations, the design of the headset frame may be used without active determination of device position. Instead, the method may use an assumption of the position of a stimulation system.

Block S124, which includes determining targeted stimulation region within a stimulation space of the stimulation system, based on the stimulation system position, functions to determine where stimulation will be targeted based on how the device is worn. The method may enable highly precise stimulation to be performed using a device with more moderate constraints on device position. As long as the targeted stimulation region is within the covered region of the plurality of electrodes, then the method can enable the system to adjust how stimulation is performed.

Block S133, which includes stimulating a subset of electrodes within a plurality of electrodes, functions to adjust effective position of stimulation by selectably using electrodes of a plurality of electrodes.

In one variation, this can include selecting an anode electrode and a cathode electrode from the plurality of electrodes such that the region between and/or surrounding the two electrodes approximates the targeted stimulation region.

In another variation, a plurality of anode electrodes may be activated with adjusted magnitude for establishing a simulated anode electrode position. Activation can include steady state activation but can additionally or alternatively include activation with temporal variation (e.g., turning on, off, or otherwise changing magnitude or polarity).

Additionally or alternatively, a plurality of cathode electrodes may be activated with adjusted magnitude for establishing a simulated cathode electrode position. Activation can include steady state activation but can additionally or alternatively include activation with temporal variation (e.g., turning on, off, or otherwise changing magnitude or polarity).

As discussed, in some variations, the method can include detecting movement of the neurostimulation device relative to the head of the subject and updating the stimulation by repeating process blocks S124 and S133 after determining the updated relative position of the device and the subject.

As discussed, a system such as the one described herein may be used to implement the method. In particular, the system may be specially configured such that processors (e.g., in the neurostimulation device and/or outside the neurostimulation device such as in connection with a management application operating on a computing device) may perform operations to perform processes of the method or variations thereof described herein.

Accordingly in one variation, a system for neurostimulation may include a neurostimulation device comprising: a headset frame with a first arc structure and a second arc structure, where the arc structure connect at opposing ends, where the first arc structures are curved and connected with angular displacement such that the first arc structure conforms to a first stimulation region of a defined head shape and the second arc structure conforms to a second stimulation region of the defined head shape; a plurality of electrodes, with a first set of electrodes integrated in the first arc structure and a second set of electrodes integrated in the second arc structure; a management application operable on a computing device; and one or more non-transitory computer-readable mediums storing instructions that, when executed by the one or more computer processors, cause a computing system to perform operations comprising: receiving stimulation input data; determining a stimulation profile based on the stimulation input data; and activating a neurostimulation device according to the stimulation profile.

The instructions may alternatively cause the computing system to perform any other operations of methods described herein. For example, the instructions may cause operations for stimulation scheduling comprising: receiving menstrual cycle data through a computer interface of the management application; analyzing the menstrual cycle data and determining a stimulation session schedule; initiating a schedule of stimulation sessions with the neurostimulation device according to the stimulation session schedule.

In a similar way, an alternative embodiment may be a non-transitory computer-readable medium storing instructions that, when executed by one or more computer processors of a computing platform, cause the computing platform to perform operations comprising: receiving stimulation input data; determining a stimulation profile based on the stimulation input data; and activating a neurostimulation device according to the stimulation profile.

The instructions may alternatively cause the computing system to perform any other operations of methods described herein. For example, the instructions may cause operations for stimulation scheduling comprising: receiving menstrual cycle data through a computer interface of the management application; analyzing the menstrual cycle data and determining a stimulation session schedule; initiating a schedule of stimulation sessions with the neurostimulation device according to the stimulation session schedule.

### 4. System Architecture

The systems and methods of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, but any suitable dedicated hardware device can (alternatively or additionally) execute the instructions.

In one variation, a system comprising of one or more computer-readable mediums (e.g., non-transitory computer-readable mediums) storing instructions that, when executed by the one or more computer processors, cause a computing platform to perform operations comprising those of the system or method described herein such as: receiving stimulation input data; determining a stimulation profile based on the stimulation input data; and activating a neurostimulation device according to the stimulation profile.

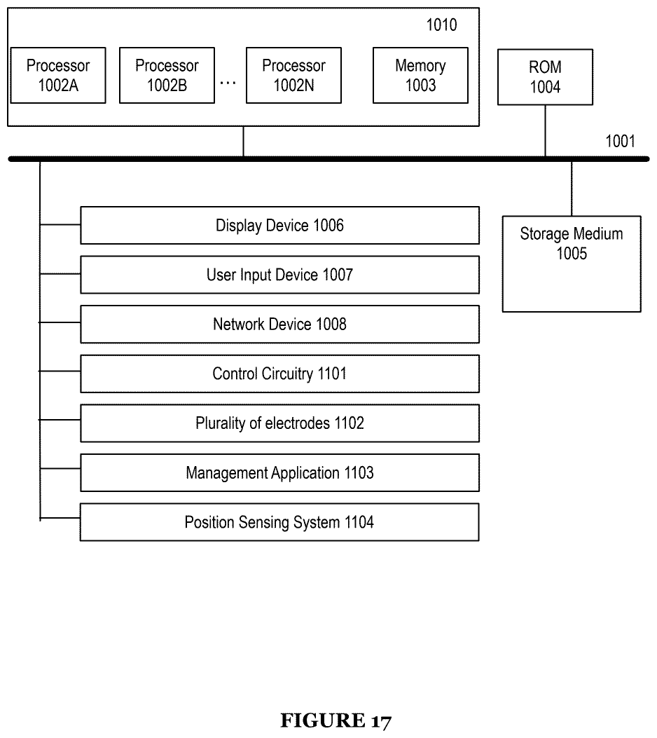

FIG. 17 is an exemplary computer architecture diagram of one implementation of the system. In some implementations, the system is implemented in a plurality of devices in communication over a communication channel and/or network. In some implementations, the elements of the system are implemented in separate computing devices. In some implementations, two or more of the system elements are implemented in same devices. The system and portions of the system may be integrated into a computing device or system that can serve as or within the system.

The communication channel 1001 interfaces with the processors 1002A-1002N, the memory (e.g., a random access memory (RAM)) 1003, a read only memory (ROM) 1004, a processor-readable storage medium 1005, a display device 1006, a user input device 1007, and a network device 1008. As shown, the computer infrastructure may be used in connecting control circuitry 1101, plurality of electrodes 1102, management application 1103, positioning sensing system 1104, and/or other suitable computing devices.

The processors 1002A-1002N may take many forms, such CPUs (Central Processing Units), GPUs (Graphical Processing Units), microprocessors, ML/DL (Machine Learning/Deep Learning) processing units such as a Tensor Processing Unit, FPGA (Field Programmable Gate Arrays, custom processors, and/or any suitable type of processor.

The processors 1002A-1002N and the main memory 1003 (or some sub-combination) can form a processing unit 1010. In some embodiments, the processing unit includes one or more processors communicatively coupled to one or more of a RAM, ROM, and machine-readable storage medium; the one or more processors of the processing unit receive instructions stored by the one or more of a RAM, ROM, and machine-readable storage medium via a bus; and the one or more processors execute the received instructions. In some embodiments, the processing unit is an ASIC (Application-Specific Integrated Circuit). In some embodiments, the processing unit is a SoC (System-on-Chip). In some embodiments, the processing unit includes one or more of the elements of the system.

A network device 1008 may provide one or more wired or wireless interfaces for exchanging data and commands between the system and/or other devices, such as devices of external systems. Such wired and wireless interfaces include, for example, a universal serial bus (USB) interface, Bluetooth interface, Wi-Fi interface, Ethernet interface, near field communication (NFC) interface, and the like.

Computer and/or Machine-readable executable instructions comprising of configuration for software programs (such as an operating system, application programs, and device drivers) can be stored in the memory 1003 from the processor-readable storage medium 1005, the ROM 1004 or any other data storage system.

When executed by one or more computer processors, the respective machine-executable instructions may be accessed by at least one of processors 1002A-1002N (of a processing unit 1010) via the communication channel 1001, and then executed by at least one of processors 1001A-1001N. Data, databases, data records or other stored forms data created or used by the software programs can also be stored in the memory 1003, and such data is accessed by at least one of processors 1002A-1002N during execution of the machine-executable instructions of the software programs.

The processor-readable storage medium 1005 is one of (or a combination of two or more of) a hard drive, a flash drive, a DVD, a CD, an optical disk, a floppy disk, a flash storage, a solid-state drive, a ROM, an EEPROM, an electronic circuit, a semiconductor memory device, and the like. The processor-readable storage medium 1005 can include an operating system, software programs, device drivers, and/or other suitable sub-systems or software.

As used herein, first, second, third, etc. are used to characterize and distinguish various elements, components, regions, layers and/or sections. These elements, components, regions, layers and/or sections should not be limited by these terms. Use of numerical terms may be used to distinguish one element, component, region, layer and/or section from another element, component, region, layer and/or section. Use of such numerical terms does not imply a sequence or order unless clearly indicated by the context. Such numerical references may be used interchangeable without departing from the teaching of the embodiments and variations herein.

As a person skilled in the art will recognize from the previous detailed description and from the figures and claims, modifications and changes can be made to the embodiments of the invention without departing from the scope of this invention as defined in the following claims.

We claim:

1. A method comprising:
receiving stimulation input data;
determining a stimulation profile based on the stimulation input data; and
activating a neurostimulation device according to the stimulation profile.

2. The method of claim 1, wherein receiving stimulation input data comprises receiving menstrual cycle data through a computer interface; wherein determining the stimulation profile based on the stimulation input data comprises analyzing the menstrual cycle data and determining a stimulation session schedule; and wherein activating the neurostimulation device according to the stimulation profile comprises initiating a schedule of stimulation sessions with the neurostimulation device according to the stimulation session schedule.

3. The method of claim 2, wherein analyzing the menstrual cycle data and determining a stimulation session schedule comprises predicting next menstrual cycle and scheduling a sequence of stimulation sessions for multiple days preceding a start of a next period in the next menstrual cycle.

4. The method of claim 3, wherein the neurostimulation device comprises a set of electrodes integrated in two distinct stimulation regions of the neurostimulation device.

5. The method of claim 4, wherein determining the stimulation session schedule comprises setting stimulation configuration for balancing stimulation across the two distinct stimulation regions.

6. The method of claim 3, comprising tracking usage of the stimulation device relative to the stimulation session schedule and updating the stimulation session schedule based on the usage.

7. The method of claim 2, wherein receiving the menstrual cycle data through a computer interface is received through user input within a user application.

8. The method of claim 2, wherein receiving the menstrual cycle data through a computer interface is received through a data interface to a third-party health data service.

9. The method of claim 2, wherein initiating a schedule of stimulation sessions with the neurostimulation device comprises triggering a session reminder through a user interface.

10. The method of claim 2, further comprising receiving stimulation feedback data and wherein determining the stimulation profile comprises updating the stimulation profile based on the stimulation feedback data.

11. The method of claim 10, wherein updating the stimulation profile based on the stimulation feedback data comprises adjusting the stimulation session schedule.

12. The method of claim 10, wherein updating the stimulation profile based on the stimulation feedback data comprises adjusting parameters defining electrical stimulation of electrodes in the neurostimulation device.

13. The method of claim 2, further comprising calibrating neurostimulation device position during a stimulation session.

14. The method of claim 13, wherein calibrating neurostimulation device position comprises activating a test stimulation and receiving user input on sensations.

15. The method of claim 13, wherein calibrating neurostimulation device position comprises performing an electrode contact test, checking positioning conditions, and generating positioning feedback.

16. The method of claim 15, wherein generating positioning feedback comprises augmenting a feedback indicator with a noise signal.

17. The method of claim 13, wherein the neurostimulator device comprises at least one array of electrodes, and wherein calibrating neurostimulation device position during a stimulation session comprises: determining neurostimulation device position relative to a head of a subject, determining targeted stimulation region within a stimulation space of the neurostimulation device, based on the stimulation system position; and stimulating a subset of electrodes within a plurality of electrodes, the subset of electrodes selected for stimulation based on the targeted stimulation region.

18. A method for neurostimulation device operation for menstrual cycle symptoms comprising:
receiving menstrual cycle data through a computer interface;
analyzing the menstrual cycle data and determining a stimulation session schedule;
initiating a schedule of stimulation sessions with neurostimulation device according to the stimulation session schedule.

19. A system comprising of:
a neurostimulation device comprising:
a headset frame with a first arc structure and a second arc structure, where the arc structure connects at opposing ends, where the first arc structures are curved and connected with angular displacement such that the first arc structure conforms to a first stimulation region of a defined head shape and the second arc structure conforms to a second stimulation region of the defined head shape,
a plurality of electrodes, with a first set of electrodes integrated in the first arc structure and a second set of electrodes integrated in the second arc structure;
a management application operable on a computing device;
one or more computer-readable mediums storing instructions that, when executed by the one or more computer processors, cause a computing platform to perform operations comprising:
receiving menstrual cycle data through a computer interface of the management application;
analyzing the menstrual cycle data and determining a stimulation session schedule;
initiating a schedule of stimulation sessions with the neurostimulation device according to the stimulation session schedule.

20. A non-transitory computer-readable medium storing instructions that, when executed by one or more computer processors of a computing platform, cause the computing platform to perform operations comprising:
receiving menstrual cycle data through a computer interface;
analyzing the menstrual cycle data and determining a stimulation session schedule;
initiating a schedule of stimulation sessions with neurostimulation device according to the stimulation session schedule.
